(19) United States
(12) Patent Application Publication
(10) Pub. No.: US 2014/0065077 A1
(43) Pub. Date: Mar. 6, 2014

(54) CONTAINER WITH CONCENTRATED SUBSTANCE AND METHOD OF USING THE SAME

(71) Applicant: Otsuka Pharmaceutical Co. Ltd., Tokyo (JP)

(72) Inventors: Scott Wayne Hollander, Princeton, NJ (US); Joel Robert Timberlake, West Windsor, NJ (US); Marjory Kadash, Skillman, NJ (US)

(21) Appl. No.: 13/934,654

(22) Filed: Jul. 3, 2013

Related U.S. Application Data
(62) Division of application No. 13/599,045, filed on Aug. 30, 2012.

(51) Int. Cl. A61J 1/00; A61K 49/04

(52) U.S. Cl. CPC A61J 1/00; A61K 49/0438
USPC 424/9.452; 424/9.454; 424/9.455

(57) ABSTRACT

Apparatus and methods are described herein for a container with a unit dose of a concentrated substance that can be diluted and orally consumed using the container. In some embodiments, an apparatus includes a container body that defines an opening in fluid communication with an interior of the container body. A cap is coupled to the container body and encloses the opening. A unit dose of a concentrated medicament or a concentrated contrast agent is disposed within the interior of the container body. The unit dose of concentrated medicament or contrast agent can be diluted to a select dilution strength with a volume of a liquid receivable through the opening and within the interior of the container body. In some embodiments, the apparatus can include a barrier member in which the container body and cap can be disposed to protect the medicament or contrast agent from light and/or moisture.

### CROSS-REFERENCE TO RELATED APPLICATIONS

This application is a divisional of U.S. patent application Ser. No. 13/599,045, filed on Aug. 30, 2012, entitled “Container With Concentrated Substance and Method of Using the Same;” the disclosure of which is herein incorporated by reference in its entirety.

### BACKGROUND

Some embodiments described herein relate to a container with a unit dose of a concentrated substance disposed therein that can receive a volume of liquid to dilute the concentrated substance to a desired concentration for oral consumption by a user.

Some known concentrated substances, such as, for example, concentrated medicaments and/or oral contrast agents, are provided to healthcare facilities in bulk containers that can hold a large quantity of the medicament or contrast agent. For example, some radiological contrast agents are typically provided in such a manner, such as those containing barium or iodine. Typically, such concentrated materials require the use of a separate container for dilution and/or consumption of the medicament or contrast agent. Such use of multiple containers for preparing a concentrated medicament or contrast agent for oral consumption can present various undesirable results, such as, for example, improper dilution strength, separation of the medicament or contrast agent from identifying labels and/or separation of the medicament or contrast agent from instructions for use.

Some known iodine based contrast media are provided as solutions that require dilution, and sometimes are provided with measuring cups to facilitate accurate dilution to different strengths. Some known ionic iodinated contrast media are available that contain flavoring, but are not provided in packaging that is ready for patient consumption. There are also some known containers that include a diluting solution in which an oral contrast agent can be mixed, but such containers still require that the active contrast agent be drawn from a large volume package. Such containers may also require drawing oral drug doses from a bottle intended and labeled for intravenous injection rather than for oral consumption. Such a situation can be undesirable, for example, if the doses are prepared in areas of a medical facility outside of the radiology suite and partially filled containers of contrast agent are kept at hand. In addition, by providing the diluting solution in such a bottle, rather than the contrast agent, such a container does not permit the range of beverage choices (flavors, carbonation, sweetness) that may lead to higher patient compliance (e.g., full consumption of the prescribed dose and concentration).

Accordingly, a need exists for a container that can include a unit dose of a concentrated substance, such as, for example, a medicament or a contrast agent, and can be used for transport, dilution and oral consumption of the concentrated substance.

### SUMMARY

Apparatus and methods are described herein for a container with a unit dose of a concentrated substance that can be diluted and orally consumed using the container. In some embodiments, an apparatus includes a container body that defines an opening in fluid communication with an interior of the container body. A cap is coupled to the container body and encloses the opening. A unit dose of a concentrated medicament or a concentrated contrast agent is disposed within the interior of the container body. The unit dose of concentrated medicament or contrast agent can be diluted to a select dilution strength with a volume of a liquid receivable through the opening and within the interior of the container body. In some embodiments, the apparatus can include a barrier in which the container body and cap can be disposed to protect the medicament or contrast agent from light and/or moisture.

### BRIEF DESCRIPTION OF THE DRAWINGS

FIG. 1 is a schematic illustration of a container with a concentrated substance, according to an embodiment.

FIG. 2 is a front perspective view of a container with a concentrated substance, according to an embodiment, shown in a first configuration.

FIG. 3 is a partially exploded side view of the container of FIG. 2, shown in a second configuration with a liquid added to the container.

FIG. 4 is a side view of the container of FIG. 2, shown in a third configuration with the concentrated substance diluted.

FIG. 5 is a side view of a container with a concentrated substance, according to another embodiment.

FIG. 6 is a front perspective view of a container with a concentrated substance, according to another embodiment.

FIG. 7 is a flow chart illustrating a method for using a container with a concentrated substance, according to an embodiment.

### DETAILED DESCRIPTION

Apparatus and methods are described herein for a container for transport and oral consumption of a unit dose of a concentrated substance, such as for example, a medicament or a contrast agent. In some embodiments, an apparatus includes a container having a container body that defines an interior volume and an opening in fluid communication with the interior volume. A cap can be removably coupled to the container body to close or cover the opening. The interior volume can contain a concentrated substance, such as, for example, a unit dose of a concentrated medicament or contrast agent. The concentrated substance can be diluted to a selected concentration or dilution strength with a volume of liquid received in the interior volume through the opening of the container body. For example, the interior volume, including the concentrated substance, can have sufficient head space to receive the volume of liquid to dilute the concentrated substance to the selected dilution strength. The apparatus can also optionally include a barrier member that can shield the contents of the container (e.g., the concentrated medicament or contrast agent) from exposure to moisture and/or light. In some embodiments, the container body itself can act as a barrier. For example, the container body can be formed with a material that can shield the concentrated substance from moisture and/or light.

In some embodiments, a method of using a container as described herein can include removing a cap from a container body. The container body can have a unit dose of a concentrated medicament or a concentrated contrast agent disposed within an interior defined by the container body. A volume of a liquid can be added to the interior of the container body such that the concentrated medicament or contrast agent is diluted to a select dilution strength. The container body can be provided to a patient to consume orally the diluted medicament or concentrated contrast agent.

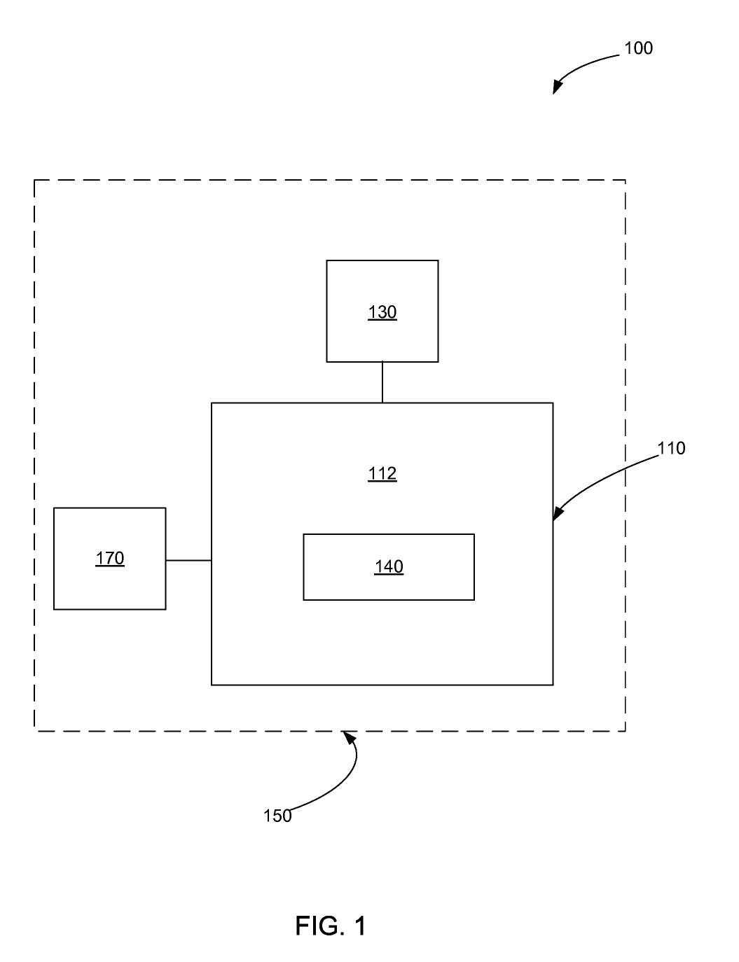

FIG. 1 is a schematic illustration of a container, according to an embodiment. A container 100 can include a container body 110 and a cap 130 that can be removably coupled to the container body 110. The container body 110 defines an interior volume 112 that can contain a concentrated substance 140 and can receive a volume of fluid to dilute the concentrated substance 140, as described in more detail below. The concentrated substance 140 can be, for example, a unit dose of a concentrated medicament or a concentrated oral contrast agent (also referred to as “contrast agent” or “oral contrast media” or “contrast media”). The container 100 can be used to store and transport the concentrated substance 140 to, for example, a healthcare facility. The container 100 can also be used at the healthcare facility to dilute the concentrated substance 140 and to provide the diluted substance to a user (e.g., a patient) for oral consumption.

The container body 110 can be a variety of different shapes, sizes and configurations. For example, the container body 110 can be in the form of a bottle, jar, tub, jug, can, and/or any other type of container. The container body 110 can have a volume of, for example, 355 ml, 500 ml, 1000 ml, 2000 ml, 20 ounces, or any other suitable volume. The container body 110 can, in some embodiments, be formed of plastic material, such as, for example, polyethylene terephthalate (PET), high density polyethylene (HDPE), low density polyethylene (LDPE), acrylic, polypropylene (PP), or any other suitable plastic. In other embodiments, the container body 110 can be formed of glass, aluminum, steel, and/or any other suitable material. The container body 110 can also be formed partially or completely with a transparent material such that the contents (e.g., the concentrated substance 140) of the container body 110 are visible. In some embodiments, the container body 110 can be formed with a material such that the container body 110 is recyclable.

The concentrated substance 140 can be provided in a variety of different forms. For example, the concentrated substance 140 can be a solid (in the form of one or more tablets, or smaller granules, powder, etc., either loose or contained in, for example, capsules or bags), a liquid, and/or any other suitable concentrate form. As described above, the concentrated substance 140 can be a concentrated medicament or a concentrated contrast agent. For example, the concentrated substance 140 can be a radiological contrast agent, such as, an iodinated contrast agent. In some embodiments, the concentrated substance 140 can be, for example, a non-ionic iodinated contrast agent, such as iohexol, iopamidol, ioversol, and/or iodixanol. In some embodiments, the concentrated substance 140 can be, for example, diatrizoate meglumine and/or diatrizoate sodium solution. In some embodiments, the concentrated substance 140 can be, for example, a monoisomer of a medicament or contrast agent, or a mixture of two or more isomers in any ratio.

In some embodiments, the concentrated substance 140 can be provided in a pure or a substantially pure form. For example, the concentrated substance 140 can be free of excipients or additives. For example, the concentrated substance 140 can be a pure or substantially pure powdered (e.g., spray dried, filter dried, milled, sifted, etc.) iohexol with a particle size of, for example, 40-50 microns or less. The dissolvability of the pure or substantially pure powdered iohexol is substantially independent of particle size. Thus, pure or substantially pure powdered iohexol can be provided in the container 100 having any suitable particle size, while still being readily and rapidly dissolvable. In one example, the pure or substantially pure powdered iohexol can be dissolved, for example, when manually agitated for between 10-20 seconds.

In an alternative embodiment, the concentrated substance 140 can be a contrast agent or medicament in the form of granules, i.e. larger particles (for example, up to 200 microns) than a powdered form. Granules may also dissolve relatively quickly, but may be more difficult to process, e.g. to dispense into container body 110 during manufacturing.

The concentrated substance 140 can be, for example, a pre-measured unit dose of a medicament or a contrast agent that can be diluted to a desired concentration strength to be orally consumed by a patient. For example, in some embodiments, the concentrated substance 140 can be approximately 9.7 g of pure or substantially pure iohexol powder that can be diluted with a liquid diluent to a selected dilution strength in the container body 110, and orally consumed from the container body 110 by a patient. In other embodiments, the concentrated substance 140 can be 13 g of pure iohexol powder or any other suitable quantity of iohexol. In other embodiments, the concentrated substance 140 can be a formulated concentrate. In some embodiments, a desired dilution strength of iohexol can be, for example, between 6 mg/ml (or can alternatively be referred to as 6 mgI/mL (grams of Iodine per milliliter)) and 25 mg/ml (or 25 mgI/mL). Similarly stated, in some embodiments, the concentrated substance 140 can contain excipients such as dispersants, disintegrants, coatings, enteric, fillers, flavors, glidants, sorbents, preservatives, sweeteners, colors, wetting agents, binders, anti-caking agents, and/or any other suitable substances to enhance dispersal, dilution, stability, taste, processability, absorption, appearance, etc. of the concentrated substance 140.

The concentrated substance 140 can occupy less than the entire interior volume 112 of the container body 110 such that a diluent can be received in the interior volume 112 to dilute the concentrated substance 140 to a desired dilution strength. The diluent can be a liquid such as, for example, water, sugar-water solution, fruit juice, milk, carbonated beverage, and/or any other suitable liquid that can be mixed with the concentrated substance 140 to dilute the concentrated substance 140 to a desired or selected dilution strength. In some embodiments, the diluent can be selected to improve palatability of the concentrated substance 140.

The container body 110 can define an opening (not shown in FIG. 1) in fluid communication (e.g., a fluid, such as a gas or a liquid, can pass between the opening and the interior volume) with the interior volume 112 of the container body 110. The cap 130 can be removably coupled to the container body 110 to close or obstruct the opening. For example, the cap 130 can be threadably coupled to a neck portion (not shown in FIG. 1) of the container body 110. When the cap 130 is removed from the container body 110, a diluent (e.g., a liquid) can be introduced into the interior volume 112 of the container 110 through the opening. The cap 130 can be, for example, a tamper-evident medicament cap, a childproof cap, and/or any other suitable type of cap that can seal the container body 110. In some embodiments, the cap 130 can be resealable. For example, the cap 130 can include one or more liners and/or seals that can form a fluid-tight seal with the container body 110 when the cap 130 is coupled thereto. The cap 130 can be formed with, for example, one or more materials, such as, for example, PP, PET, HDPE, LDPE, aluminum, steel, and/or any other suitable material(s).

The container 100 can optionally include a label 170. The label 170 can be coupled to the container body 110 and provide information, such as, for example, information about the contents of the container 100. The label 170 can be coupled to the container body 110 with, for example, an adhesive. In some embodiments, the label 170 can be coupled to the container body 110 with an adhesive that allows the label 170 to be removable or peelable with limited or no damage to the label 170. For example, it may be desirable to remove the label 170 from the container body 110 and couple the label 170 to another object or item, such as, for example, to a patient record or chart. In other embodiments, the label 170 can be coupled to and/or provided within an exterior packaging, such as a barrier member 150 (described in further detail below), or other exterior packaging.

In some embodiments, such as embodiments in which the concentrated substance 140 is a medicament or contrast agent, the label 170 can contain drug and/or regulatory information. The label 170 can also include use instructions, such as instructions regarding the amount of diluent to add to the container body 110 to obtain a desired dilution strength, instructions regarding the mixing of the concentrated substance 140 and the diluent, and/or instructions related to the consumption of the contents of the container body 110. For example, different dilution strengths may be desired for different types of the concentrated substance 140 and/or for different uses of the concentrated substance 140. For example, a dilution strength for a contrast agent for use in imaging of an upper gastrointestinal (GI) tract may be different than a dilution strength for use of a contrast agent for imaging of a lower portion of the GI tract.

In some embodiments, the label 170 can include one or more markings or indications (not shown in FIG. 1) that can be used by a medical professional or the patient to measure the desired amount of diluent to add to the container body 110 to obtain a desired dilution strength. For example, the label 170 can contain markings located adjacent to a transparent portion of the container body 110. The markings can include, for example, volumetric measurement graduations such as graduations in milliliters (ml), or ounces, and/or the markings can include concentration measurement graduations, such as graduations in milligrams per milliliter (mg/ml) (e.g., milligrams of concentrated substance and/or active ingredient per milliliter), etc. In some embodiments, the markings can include a combination of different types of markings or indications. For example, the label can include markings associated with fill volume, for example, in milliliters, and parallel markings associated with concentration strengths, for example, in milligrams per milliliter. In some embodiments, the markings can be indicators that identify one or more fill levels that correspond to one or more specified dilution strengths rather than actual measurement graduations (e.g., ml).

In some embodiments, instructions can be provided that instruct the user to add diluent to a particular indicator that corresponds to a particular dilution strength (e.g., mg/ml). In other embodiments, the label 170 can include directions instructing a user to add a volume of diluent to the container body 110 to obtain a specified dilution strength and/or amount of diluted concentrated substance 140. For example, the label 170 can include directions to instruct a user to add a pre-measured volume of diluent to the container body 110 based on the amount of concentrated substance 140 disposed within the container body 110 such that a desired volume and concentration strength of the diluted concentrated substance 140 can be obtained for consumption by a user. In an alternative embodiment, the container body 110, rather than the label 170, can include markings or indications that can be used to prepare the concentrated substance 140 for consumption by a user. For example, the container body 110 can have markings imprinted, formed, molded, engraved, etc., into the material of the container body 110, or the markings can be engraved or printed on the container body 110. In some embodiments, the label 170 can include a portion that can be used by a medical professional or a patient to add a notation(s), such as, for example, the patient name, type of drug, date of preparation, dosage administered, etc. In some embodiments, both the container body 110 and the label 170 can include markings.

In some embodiments, the container body 110 can be stored and/or transported within an optional barrier member 150. The barrier member 150 can be a variety of different shapes, sizes and/or configurations. For example, the barrier member 150 can be a pouch or bag, a box, or other suitable packaging form. The barrier member 150 can be formed with, for example, a material or materials that can reduce or eliminate exposure of the concentrated substance 140 to moisture and/or light. For example, the barrier member 150 can be, formed with, for example, a polymer-aluminum laminate. The barrier member 150 can be, for example, hermetically sealed via ultrasonic welding, heat sealing, and/or any other suitable sealing mechanism. Such a barrier member 150 can be desirable, for example, for storage and/or transport of certain concentrated medicaments and/or contrast agents, which are sensitive to light and/or moisture. For example, substances such as a pure or substantially pure iohexol can be sensitive to moisture and/or light. For example, exposure to moisture over a time period can result in clumping or agglomeration of the particles of iohexol, which can affect the effectiveness and/or usability of the iohexol. In some cases, agglomeration can occur, for example, in several days to several weeks. Thus, if the container body 110 is formed with a material that is not sufficiently impermeable to light and/or moisture (e.g., certain PET materials) and contains a concentrated substance such as a hygroscopic monoisomer of iohexol, it may be desirable to place the container 100 within such a barrier member 150.

In other embodiments, the container body 110 can be formed with a material that is sufficiently impermeable to moisture and/or light such that the concentrated substance 140 is sufficiently protected by the container alone. In such an embodiment, the container 100 can be stored and transported without a separate barrier member 150. For example, in some embodiments, the container body 110 can be formed with an opaque material and/or a material with low permeability, such that the container body 110 can prevent light and/or moisture from passing through the container body 110. In some embodiments, in addition to the container body 110 and/or a barrier member 150, or alternatively, the label 170 can provide protection to the concentrated substance 140 within the container body 110. For example, the label 170 can be opaque to prevent light from passing through the container body 110 and/or the label 170 can include a low-permeability component, such as a foil backing. The label 170 can be bonded to the container 110 such that exposure of the concentrated substance 140 to light and/or moisture can be reduced or eliminated. For example, the label 170 can be sized such that, when applied to the container body 110, the label 170 covers substantially all or a portion of the surface of the container body 110.

As described above, the container 100 can be used to store and transport the concentrated substance 140 (e.g., a concentrated medicament or contrast agent) and can also be used during the dilution, mixing and consumption of the concentrated substance 140. Thus, the use of other containers and/or measuring devices can be reduced or eliminated. For example, the container 100 can be delivered to a medical facility and a medical professional can remove the cap 130 from the container body 110 and add a desired amount of diluent to the container body 110 and then replace the cap on the container body 110 to reseal the container body 110. The user can obtain the desired dilution strength by adding the diluent to an appropriate marking on the label 170 or container body 112, as described above. The medical professional can then agitate (e.g., either manually or mechanically) the container 100 such that the diluent is mixed with the concentrated substance 140 and dilutes and/or dissolves the concentrated substance 140. For example, as described above, when the concentrated substance 140 is a pure or substantially pure powder iohexol, the concentrated substance 140 can be readily and rapidly dissolved through manual agitation by a user. The medical professional can then provide the container 100 to a patient (with or without the cap 130 coupled thereto) so that the patient can orally consume the diluted substance using the container body 112. For example, in some embodiments, the concentrated substance 140 can be a concentrated contrast agent, and the diluted contrast agent can be provided to a patient for oral consumption prior to an imaging procedure.

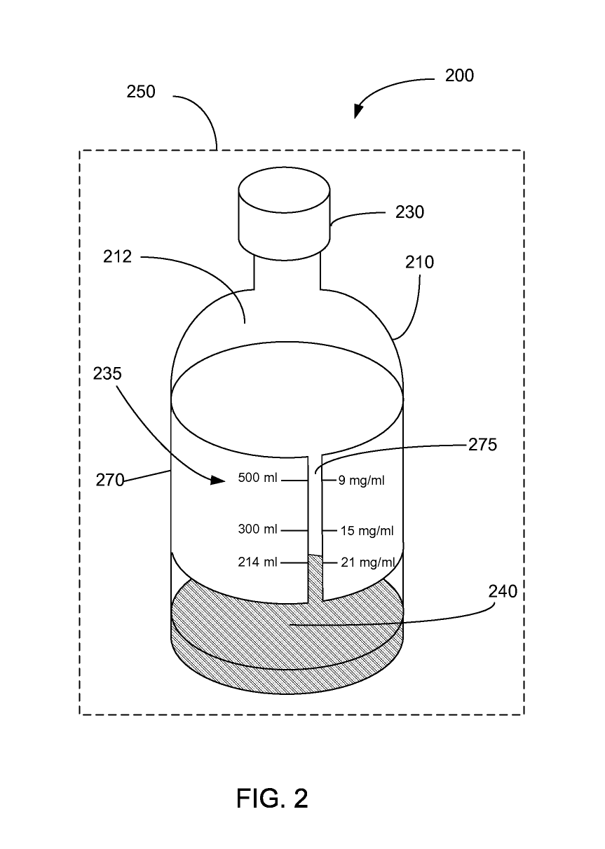

FIGS. 2-4 illustrate a container, according to another embodiment. A container 200 includes a container body 210 and a cap 230 that can be removably coupled to the container body 210. The container 200 can be functionally and/or structurally the same as or similar to the container 100 described above with reference to FIG. 1. The container body 210 defines an interior volume 212 and an opening 220 in fluid communication with the interior volume 212. A concentrated substance 240 is disposed within the interior volume 212 of the container body 210. The cap 230 can include a seal and/or liner to hermetically seal the interior volume 212 and include a threaded portion (not shown) that can be threadably coupled to a threaded portion 236 of the container body 210. The container 200 can also include a label 270 coupled to the container body 210.

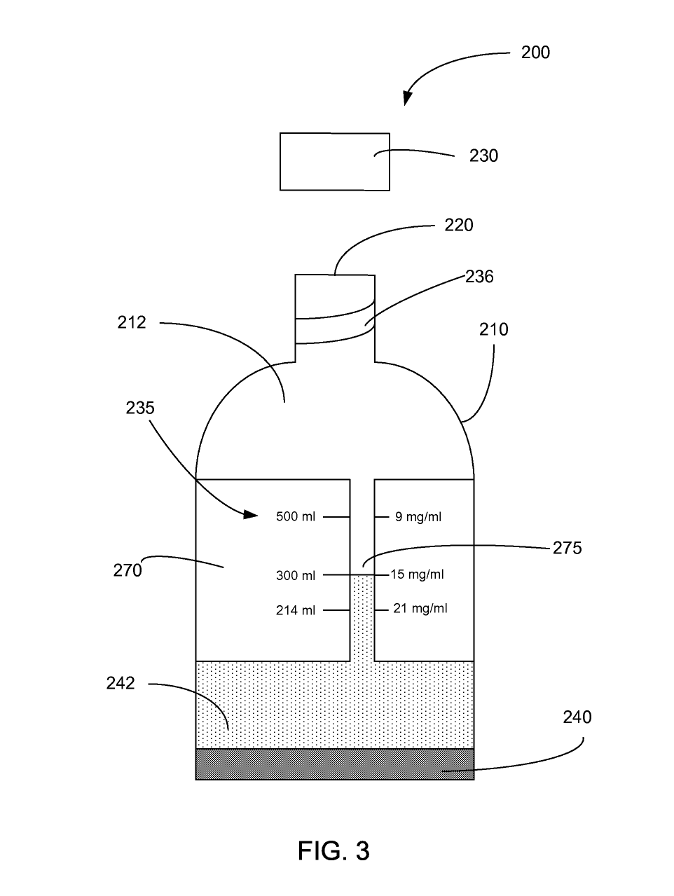

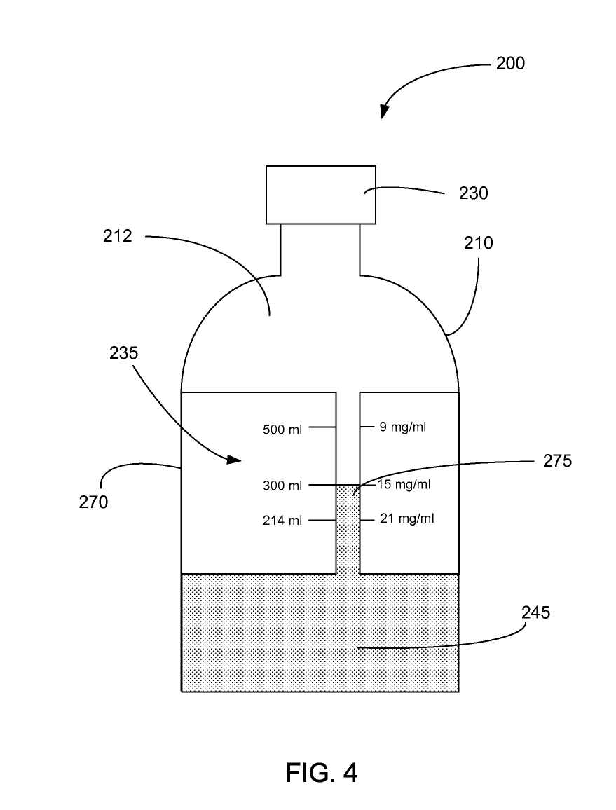

FIG. 2 depicts the container 200 in a first configuration, during storage and/or transport, in which the cap 230 is coupled to the container body 212 and the container 200 is disposed within a barrier member 250. FIG. 3 depicts the container 200 in a second configuration, in which the cap 230 is removed from the container body 210 and a diluent 242 (e.g., water or flavored liquid) has been added to the container body 210, and FIG. 4 depicts the container 200 in a third configuration, in which the concentrated substance 240 has been diluted or dissolved to form a diluted substance 245.

In this embodiment, the concentrated substance 240 is in the form of a concentrated powder. As shown in FIG. 2 the quantity of concentrated substance 240 is such that the interior volume 212 includes sufficient head space to allow for the addition of a volume of diluent 242 (e.g., water) to dilute or dissolve the concentrated substance 240 to a select dilution strength, as described in more detail below. The concentrated substance 240 can be, for example, a unit dose of a concentrated medicament or a concentrated contrast agent as described above.

The barrier member 250 can be, for example, in the form of an aluminum-polymer laminar bag in which the container 200 can be disposed during storage and transport as described above with reference to FIG. 1. The barrier member 250 can sealably enclose the container 200 to shield and/or protect the container 200 and its contents (e.g., concentrated substance 240) from moisture and/or light. For example, as shown in FIGS. 2-4, the container body 210 can be formed with a transparent material and the barrier member 250 can prevent the concentrated substance 240 from being exposed to light.

The label 270 can be sized such that a window 275 is defined through which a user (e.g., a medical professional or patient) can view a fill level of the interior volume 212 as the diluent 242 is added to the container body 210. The label 270 can include one or more measurement markings or indications 235 that can be used to measure an amount of diluent to add to the container body 210 to obtain a desired dilution strength and/or volume of a diluted substance 245. The markings 235 can include, for example, volumetric graduations such as graduations in milliliters, ounces, etc., and concentration levels corresponding to the volumetric graduations as shown in FIGS. 2-4, and/or other indicators. The type and quantity of markings 235 shown in FIGS. 2-4 are just an example of the type and quantity of markings that can be used and are not to scale. It should be understood that other types and quantities of markings can alternatively be used. For example, the type and quantity of markings can depend on the particular concentrated substance 240.

In use, a user (e.g., healthcare professional or patient) can open the barrier member 250 (e.g., cut or tear the bag or pouch) and remove the container 200 from the barrier member 250. The cap 230 can then be removed from the container body 210, exposing the opening 220. The user can then add a volume of diluent 242 through the opening 220 and fill the container body 210 to a desired marking 235 as shown in FIG. 3. In this example, the volume of liquid has been added to the marking 300 ml, which in this example corresponds to a concentration of 15 mg/ml. In other embodiments, the user can add a different volume of diluent to the container body 210 to obtain a different concentration strength. The user can then recouple the cap 230 to the container body 210 and agitate the container 200 to mix the diluent 242 and the concentrated substance 240. After the concentrated substance 240 has been diluted it will be the diluted substance 245, as shown in FIG. 4. The container 200 can then be provided to a patient, either with or without the cap 230 coupled to the container body 212, so that the patient can consume the diluted substance 245 orally. For example, in some embodiments, the concentrated substance is a unit dose of a concentrated contrast agent (e.g., iohexol), and the diluted contrast agent can be provided to a patient to consume orally prior to an imaging procedure.

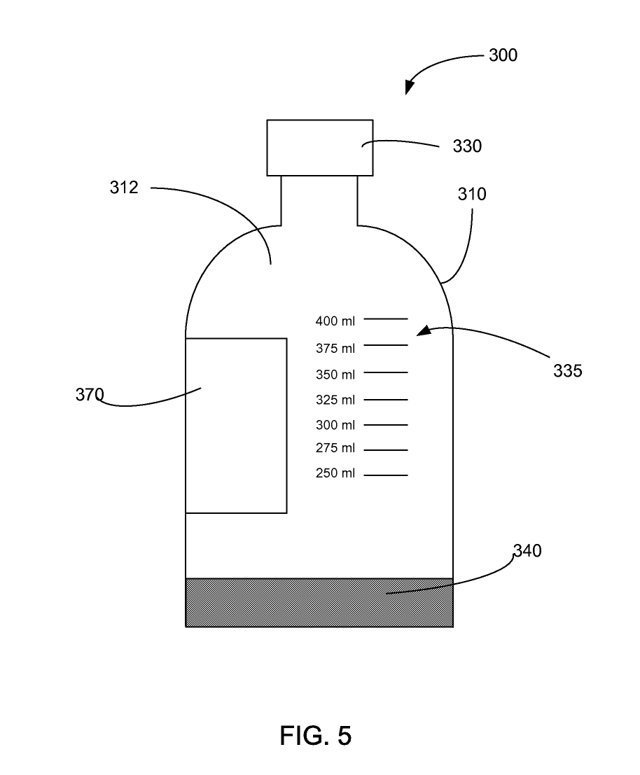

FIG. 5 illustrates a container, according to another embodiment. A container 300 includes a container body 310 and a cap 330 that can be removably coupled to the container body 310 in the same or similar manner as described above for previous embodiments. The container 300 can be functionally and/or structurally the same as, or similar to, the containers 100 and 200 described above. The container body 310 defines an interior volume 312 that can contain a concentrated substance 340 therein and an opening (not shown in FIG. 5) in fluid communication with the interior volume 310. In this embodiment, the concentrated substance 340 is in the form of a concentrated liquid that can be mixed and diluted with the addition of a diluent. The concentrated substance 340 can be for example, a concentrated medicament or a concentrated oral contrast agent. The container 300 also includes a label 370 that can include information about the concentrated substance 340 and directions for its use, as described previously. Although not shown, the container 300 can also be disposed within a barrier member during transport of the container 300 to protect and/or shield the container 300 and its contents from moisture and/or light, as described above for previous embodiments.

In this embodiment, the container body 310 can be formed with a transparent material as with the previous embodiment, and can include multiple markings or indications 335 that can be used to measure the desired amount of diluent to add to the container body 310. For example, the markings 335 can be molded into the material in which the container body 310 is formed, or can be printed on the container body 310, or coupled to the container body 310 by other methods. The container 300 can be used in the same manner as described above for previous embodiments, to store and transport a concentrated substance (e.g. a concentrated medicament or contrast agent), dilute the concentrated substance to a desired dilution strength, and to be provided to a patient to consume the diluted substance orally.

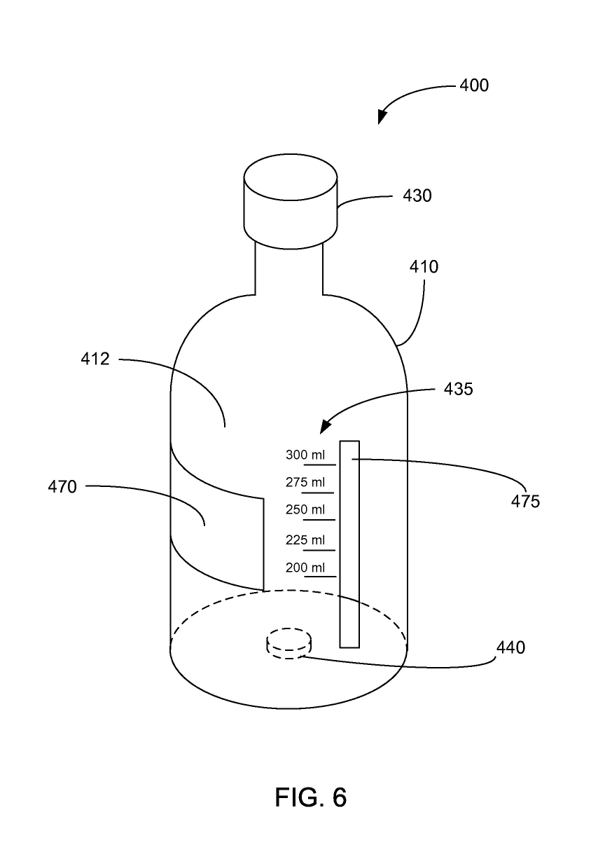

FIG. 6 illustrates a container, according to another embodiment. A container 400 includes a container body 410 and a cap 430 that can be removably coupled to the container body 410 in the same or similar manner as described above for previous embodiments. The container 400 can be functionally and/or structurally the same as, or similar to, the containers 100, 200 and 300 described above. The container body 410 defines an interior volume 412 that can contain a concentrated substance 440 therein and an opening (not shown in FIG. 6) in fluid communication with the interior volume 412. In this embodiment, the concentrated substance 440 is in the form of a tablet that can be dispersed and/or dissolved by the addition of a diluent. The container 400 also includes a label 470 that can include information about the concentrated substance 440 and directions for its use, as described previously.

In this embodiment, the container body 410 is formed with a material that can protect or shield the concentrated substance from moisture and/or light without the use of a separate barrier member (e.g., 150, 250 described above). For example, the container body 410 can be formed with an opaque material having low permeability, for example, a high density polyethylene (HDPE), or a low density polyethylene (LDPE) material. The container body 410 includes a window 475 to allow a user (e.g., healthcare professional) to view at least a portion of the interior volume 412 of the container body 410. For example, the window 475 can be a transparent portion of the container body 410. In alternative embodiments, the container body may be formed with a low permeability material that provides sufficient visibility through the material of the container body to view the contents such that the container body does not need a window. The container body 410 also includes one or more markings or indications 435 that can be used to measure an amount of diluent to add to the container body 410 as described above. Thus, a user can add a volume of diluent to the container body 410 and view the level of the diluent being added through the window 475 and use the markings 435 to measure the desired amount of diluent. The container 400 can be used in the same manner as described above for previous embodiments, to store and transport a concentrated substance (e.g., a concentrated medicament or contrast agent), dilute the concentrated substance to a desired dilution strength, and to be provided to a patient to orally consume the diluted substance.

In an alternative embodiment, the concentrated substance can be contained within the cap of the container, rather than in the interior volume defined by container body. For example, the cap can define a compartment or interior region that can contain the concentrated substance. For example, in such an embodiment, the cap can include a seal that can be punctured or removed by the user to allow the concentrated substance to be expelled out of the interior region of the cap and into the interior volume of the container body. The container can then be used in the same manner as described above for other embodiments to add a volume of diluent to dissolve or dilute the concentrated substance within the container body and such that a patient can orally consume the diluted substance using the container body.

In an alternative embodiment, the concentrated substance can be contained in an inner container, such as a packet, bag, sachet, etc. that is permeable to the diluents and to the diluted substance. In such an embodiment, when the diluent is added to the container, it permeates the inner container, diluting the concentrated substance. The diluted substance can be released from the inner container.

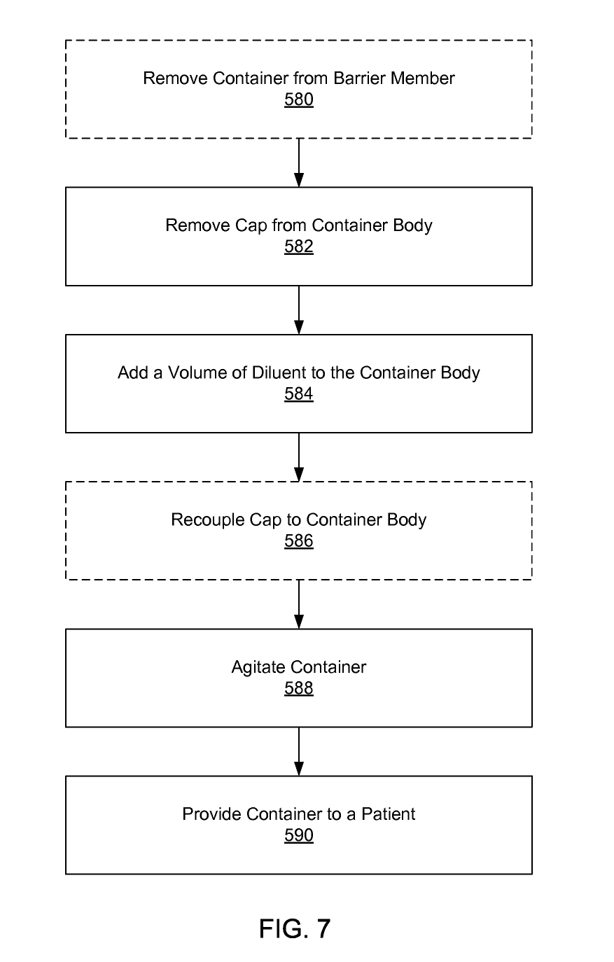

FIG. 7 is a flowchart illustrating a method of using a container including a concentrated substance, such as, for example, the containers (e.g., 100, 200, 300, 400) described herein. Optionally at 580, a user can remove a container (e.g., 100, 200, 300, 400) from a barrier member (e.g., 150, 250). For example, the container can be disposed within a barrier member as described herein that can protect or shield the concentrated substance from being exposed to light and/or moisture. In an embodiment in which the barrier member is in the form of a bag or pouch, the user can, for example, cut or tear the bag or pouch and remove the container. The container can include a container body (e.g., 110, 210, 310, 410) and a cap (e.g., 130, 230, 330, 430) coupled thereto enclosing an interior of the container body as described herein. The container body can have a unit dose of a concentrated substance, such as, for example, a concentrated medicament or a concentrated contrast agent, disposed within its interior.

The user can remove the cap from the container body at 582, exposing an opening defined by the container body. At 584, the user can add a volume of a diluent to the container body via the opening. For example, the user can add a diluent, such as water, to the container to dilute the concentrated substance to a select dilution strength. For example, as described herein, instructions on how to dilute the concentrated substance can be provided with the container. In some embodiments, the instructions can be provided on a label coupled to the container body. In some embodiments, the container body can include one or more markings or indications to use to measure the volume of diluent to be added. The markings can be provided, for example, on the label or on the container body

After adding the volume of diluent (e.g., water) to the container body, at 586, the user can optionally replace the cap, thereby sealing the container body. At 588, the user can agitate the container, for example, by manually shaking or agitating the container. Agitating the contents can aid in the dispersal and/or dissolution of the concentrated substance into the diluent to provide a homogenous diluted substance. At 590, the container can be provided to a patient so that the diluted substance can be orally consumed by the patient. The container can be provided to the patient with or without the cap couple to the container body. For example, in some embodiments, the concentrated substance can be a concentrated contrast agent, and the diluted contrast agent can be provided to a patient prior to an imaging procedure.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made. For example, although various embodiments have been described as having particular features and/or combinations of components, other embodiments are possible having any combination or sub-combination of any features and/or components from any of the embodiments described herein.

1. A method, comprising:
removing from an interior of a barrier member a container including a container body and a cap coupled to the container body, the container body having a unit dose of an excipient free concentrated contrast agent in powder form disposed within an interior of the container body, the barrier member configured to shield the unit dose of excipient free concentrated contrast agent from at least one of light or moisture;
removing the cap from the container body;
after the removing, adding a volume of a liquid to the interior of the container body such that the unit dose of the excipient free concentrated contrast agent is diluted to a select dilution strength; and
after the adding, providing the container body to a patient to orally consume the diluted excipient free concentrated contrast agent.

2. The method of claim 1, wherein the container body is provided to the patient prior to an imaging procedure to be performed on the patient.

3. The method of claim 1, further comprising:
after the adding the volume of liquid, coupling the cap to the container body; and
agitating the container body such that the volume of liquid is mixed with the excipient free concentrated contrast agent.

4. The method of claim 1, further comprising:
after the adding the volume of liquid, coupling the cap to the container body;
agitating the container body such that the volume of liquid is mixed with the excipient free concentrated contrast agent; and
removing the cap from the container body prior to providing the container body to the patient.

5. The method of claim 1, further comprising:
after the adding the volume of liquid, coupling the cap to the container body; and
agitating the container body such that the volume of liquid is mixed with the excipient free concentrated contrast agent prior to providing the container body to the patient.

6-8. (canceled)

9. A method, comprising:
providing a container having a container body defining an opening in fluid communication with an interior of the container body and a cap sealed to the container body and enclosing the opening;
removing the cap from the container body, the container body having a unit dose of an excipient free concentrated contrast agent disposed within an interior defined by the container body;
adding a volume of a liquid to the interior of the container body such that the unit dose of the excipient free concentrated contrast agent is diluted to a select dilution strength; and
after the adding, providing the container body to a patient to orally consume the diluted excipient free concentrated contrast agent.

10. The method of claim 9, wherein the container body is provided to the patient prior to an imaging procedure to be performed on the patient.

11. The method of claim 9, further comprising:
after the adding the volume of liquid, coupling the cap to the container body; and
agitating the container body such that the volume of liquid is mixed with the excipient free concentrated contrast agent.

12. The method of claim 9, further comprising:
after the adding the volume of liquid, coupling the cap to the container body;
agitating the container body such that the volume of liquid is mixed with the excipient free concentrated contrast agent; and
removing the cap from the container body prior to providing the container body to the patient.

13. The method of claim 9, further comprising:
after the adding the volume of liquid, coupling the cap to the container body;
agitating the container body such that the volume of liquid is mixed with the excipient free concentrated contrast agent prior to providing the container body to the patient.

14. The method of claim 9, further comprising:
prior to the removing the cap, removing the container body with the cap disposed thereon from an interior of a barrier member, the barrier member being configured to shield the unit dose of the excipient free concentrated contrast agent from at least one of light or moisture.

15. A method, comprising:
removing a cap from a container body, the container body having a unit dose of a dilutable powder iodinated contrast agent disposed within an interior defined by the container body;
adding a volume of a liquid to the interior of the container body such that the interior of the container body is substantially filled and the unit dose of the powder iodinated contrast agent is diluted to a select dilution strength of no more than 6 mgI/ml; and
after the adding, providing the container body to a patient to orally consume the diluted powder iodinated contrast agent.

16. The method of claim 15, wherein the container body is provided to the patient prior to an imaging procedure to be performed on the patient.

17. The method of claim 15, further comprising:
after the adding the volume of liquid, coupling the cap to the container body; and
agitating the container body such that the volume of liquid is mixed with the unit dose of a dilutable powder contrast agent.

18. The method of claim 15, further comprising:
after the adding the volume of liquid, coupling the cap to the container body;
agitating the container body such that the volume of liquid is mixed with the unit dose of a dilutable powder contrast agent; and
removing the cap from the container body prior to providing the container body to the patient.

19. The method of claim 15, further comprising:
after the adding the volume of liquid, coupling the cap to the container body;
agitating the container body such that the volume of liquid is mixed with the unit dose of a dilutable powder contrast agent prior to providing the container body to the patient.

20. The method of claim 15, further comprising:
prior to the removing the cap, removing the container body with the cap disposed thereon from an interior of a barrier member, the barrier member being configured to shield the unit dose of a dilutable powder contrast agent from at least one of light or moisture.

21-22. (canceled)
